(19) United States
(12) Patent Application Publication
(10) Pub. No.: US 2024/0148124 A1
(43) Pub. Date: May 9, 2024

(54) NAIL TOOL FOR REMOVING TISSUE IN NAIL GROOVE

(71) Applicant: Ching-Lu CHANG, Taipei City (TW)

(72) Inventor: Ching-Lu CHANG, Taipei City (TW)

(21) Appl. No.: 18/501,339

(22) Filed: Nov. 3, 2023

(30) Foreign Application Priority Data
Nov. 4, 2022 (TW) 111142254

(51) Int. Cl. A45D 29/16

(52) U.S. Cl. CPC A45D 29/16

(57) ABSTRACT

A nail tool for removing tissue in a nail groove is adapted for extending into the nail groove to remove the tissue. The nail tool includes a rod body assembly and a tissue removing member. The rod body assembly extends in a front-rear direction and includes a rod body. The rod body has a front end portion, and a rear end portion that is located opposite to the front end portion. The tissue removing member is connected to the front end portion of the rod body, and includes a seat body and a removing section that is located at a front end of the seat body. The seat body has a groove wall that defines an accommodating groove. The removing section has a rough surface. The groove wall is adapted to widen the nail groove to allow the rough surface to remove the tissue.

### CROSS-REFERENCE TO RELATED APPLICATION

This application claims priority to Taiwanese Invention Patent Application No. 111142254, filed on Nov. 4, 2022.

### FIELD

The disclosure relates to a nail tool, and more particularly to a nail tool for removing tissue in a nail groove.

### BACKGROUND

In general, ingrown nail commonly happens on a toe, especially the big toe. If the ingrown nail is not treated in time, a person suffered from the ingrown nail may first feel pain, and the pain may increase when skin near the ingrown nail becomes red and swollen which is known as paronychia, and cellulitis may occur if paronychia is not treated. Therefore, the person usually treats the ingrown nail himself/herself at home by using a nail clipper to clip the nail that grows into the skin to relieve the discomfort caused by the ingrown nail. However, since conventional nail tools (for example, nail clipper, nail scissor, nail plier) having large sizes may not be able to extend deep into a nail groove to completely remove the nail that grows into the skin and may cause pain while doing so, the ingrown nail may not be treated completely.

### SUMMARY

Therefore, an object of the disclosure is to provide a nail tool for removing tissue in a nail groove that can alleviate at least one of the drawbacks of the prior art.

According to the disclosure, the nail tool is adapted for extending into the nail groove to remove the tissue. The nail tool includes a rod body assembly and a tissue removing member. The rod body assembly extends in a front-rear direction and includes a rod body. The rod body has a front end portion and a rear end portion that is located opposite to the front end portion. The tissue removing member is connected to the front end portion of the rod body, and includes a seat body and a removing section that is located at a front end of the seat body. The seat body has a groove wall that defines an accommodating groove. The removing section has a rough surface. The groove wall is adapted to widen the nail groove to allow the rough surface to remove the tissue.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a fragmentary side view of a first embodiment of a nail tool for removing tissue in a nail groove according to the disclosure.

FIG. 2 is an exploded perspective view of the first embodiment.

FIG. 3 is a top view of a tissue removing member of the first embodiment.

FIG. 4 is a fragmentary sectional view of the first embodiment.

FIG. 5 is a schematic view of a manufacturing process of the tissue removing member of the first embodiment.

FIG. 6 is a fragmentary enlarged side view of the tissue removing member of another example of the first embodiment.

FIG. 7 is a fragmentary sectional view of a second embodiment of the nail tool of the disclosure.

FIG. 8 is an exploded perspective view of a tissue removing member of the second embodiment.

FIG. 9 is a fragmentary top view of a third embodiment of the nail tool of the disclosure.

FIG. 10 is a perspective view of a tissue removing member of the third embodiment.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

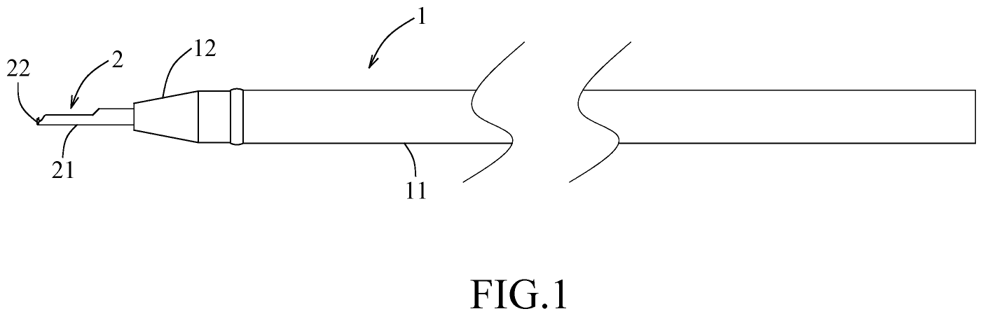

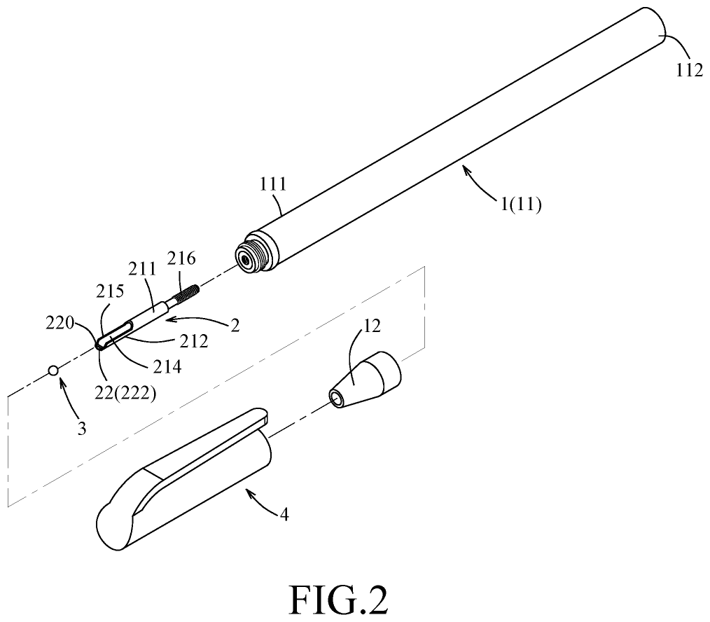

Referring to FIGS. 1 and 2, a first embodiment of a nail tool for removing tissue in a nail groove according to the disclosure is illustrated. The nail tool is adapted for extending into the nail groove to remove the tissue. The nail tool includes a rod body assembly 1 and a tissue removing member 2. The tissue includes, but are not limited to, nail, broken nail, and skin.

The rod body assembly 1 extends in a front-rear direction, and includes a rod body 11 having a front end portion 111 and a rear end portion 112 that is located opposite to the front end portion 111.

The tissue removing member 2 is connected to the front end portion 111 of the rod body 11, and includes a seat body 21 and a removing portion 22.

Referring to FIGS. 2, 3, 4 and 6, the seat body 21 has a connecting section 211 that is connected to the rod body 11, and a widening section 212 that is located at a front end of the seat body 21 and that is connected to an end of the connecting section 211 opposite to the rod body 11. The connecting section 211 defines an accommodating space 213. The widening section 212 has a groove wall 215. The groove wall 215 defines an accommodating groove 214 and is adapted to widen the nail groove. The accommodating groove 214 is in spatial communication with the accommodating space 213. The groove wall 215 has a bottom wall portion 215a, and two side wall portions 215b that extend from the bottom wall portion 215a.

In this embodiment, the removing portion 22 is connected to the front end portion 111 of the rod body 11, and has a removing section 222 that is located at the front end of the seat body 21 and that has a rough surface 220 for removing the tissue, and an extension section 221 that is connected between the widening section 212 of the seat body 21 and the removing section 222. The removing portion 22 extends forwardly from the bottom wall portion 215a of the groove wall 215 and is bent back to form a hook shape.

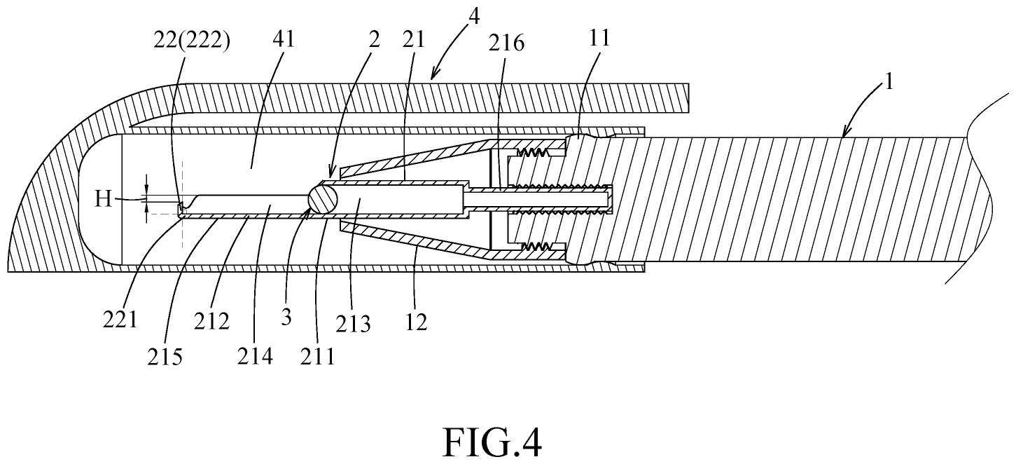

Furthermore, referring to FIG. 4, in this embodiment, the groove wall 215 has an open end, and a closed end that is opposite to the open end in a transverse direction perpendicular to the front-rear direction. A distance between a distal end of the removing section 222 that is distal from the rod body 11 and the closed end of the groove wall 215 in the transverse direction is smaller than a distance between the open end and the closed end of the groove wall 215 in the transverse direction. Specifically, a distance (H) between the distal end of the removing section 222 and the open end of the groove wall 215 in the transverse direction ranges from 0.1 mm to 1.0 mm. In some examples of the embodiment, the distance (H) ranges from 0.2 mm to 0.5 mm.

When using the nail tool, the user operates the nail tool to extend into the nail groove using the removing portion 22 and then widen the nail groove by the side wall portions 215b of the groove wall 215 so that the removing section 222 may further extend into the nail groove. Next, the user operates the nail tool to remove the tissue by sliding the removing section 222 along the tissue, and the tissue thus removed enters the accommodating groove 214 from the front end of the accommodating groove 214. After the removing section 222 moves along the tissue several times, the tissue may be completely removed. Before using the nail tool, the user may take a bath or soak his/her finger/toe where the tissue to be removed is located so as to soften the tissue, thereby facilitating removal of the tissue and reducing pain during the removal.

In this embodiment, the nail tool may further include a plug 3 that is disposed in the accommodating groove 214. The plug 3 is adapted to prevent the tissue thus removed from entering the accommodating space 213 through the accommodating groove 214, thereby making cleaning of the nail tool easier.

In the embodiment, as shown in FIGS. 2 to 4, the seat body 21 further has a threaded section 216 that is located at a rear end of the seat body 21 and that is screwed to the front end portion 111 of the rod body 11, so that the tissue removing member 2 is detachably connected to the rod body 11 by screwing through the threaded section 216. It should be noted that the detachable connection between the tissue removing member 2 and the front end portion 111 of the rod body 11 may be implemented by, but not limited to, engaging, squeezing, magnetic attraction, clamping, etc.

In addition, the rod body assembly 1 may further include a protecting member 12 that is connected to the rod body 11, and that surrounds and covers a portion of the tissue removing member 2. Therefore, the protecting member 12 may improve a connection strength between the tissue removing member 2 and the rod body 11. Specifically, as shown in FIG. 4, the protecting member 12 may cover the threaded section 216 and the front end portion 111 such that separation of the threaded section 216 from the front end portion 111 may be prevented.

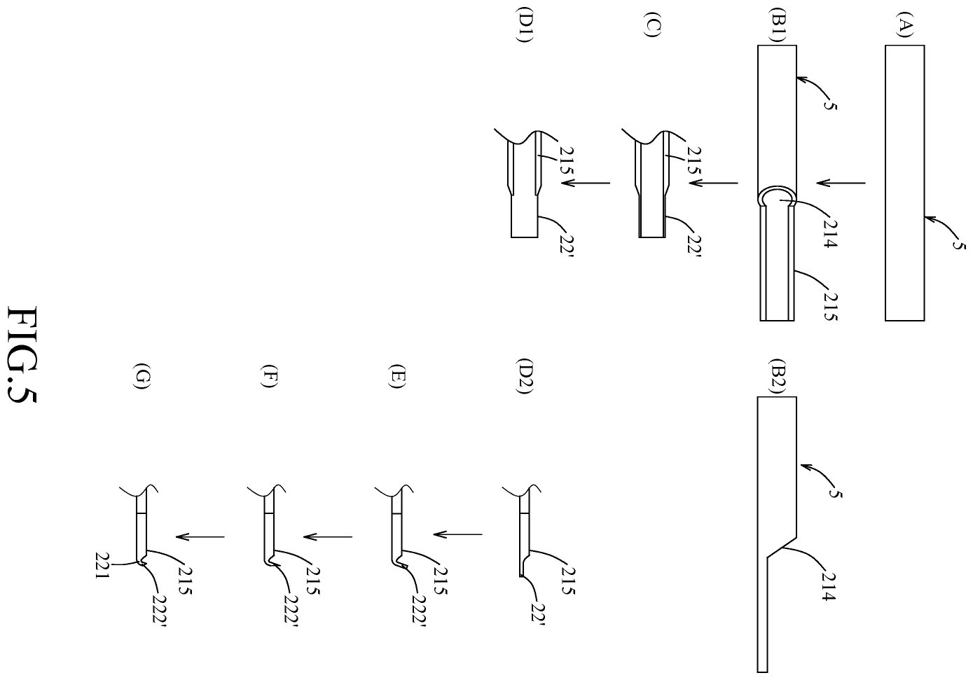

Moreover, referring to FIG. 5, in this embodiment, the tissue removing member 2 may be integrally formed, which is formed by a tubular piece 5. The tubular piece 5 is, for example, a capillary tube made of stainless steel, which is resistant to rust and corrosion and which is highly rigid, and having a thickness ranging from 0.5 mm to 3.0 mm. The tubular piece 5 may also be made of tin, iron, copper, or other high rigid metal, such that the tissue removing member 2 is capable of being used repeatedly. FIG. 5 illustrates a manufacturing process of the tubular piece 5, which includes steps shown in Table 1 below.

TABLE 1

| | Top | Side |
|---|---|---|
| Steps | views | views |
| Preparing the tubular piece 5 | (A) | |
| Cutting the tubular piece 5 to form the | (B1) | (B2) |
| accommodating groove 214 | | |
| Cutting the front end of the groove wall 215 to form | (C) | |
| an unfinished removing portion 22′ | | |
| Flattening the unfinished removing portion 22′ | (D1) | (D2) |
| Initially bending the unfinished removing portion 22′ | | (E) |
| to form an unfinished removing section 222′ | | |
| Cutting the unfinished removing section 222′ | | (F) |
| Bending the removing section 222′ to form the | | (G) |
| removing section 222 | | |

First, an end of the tubular piece 5 is cut to form the accommodating groove 214. Thereafter, the groove wall 215 that defines the accommodating groove 214 is cut in a radial direction to form a tapered unfinished removing portion 22′. Subsequently, the unfinished removing portion 22′ is flatten, and then the front end of the unfinished removing portion 22′ is initially bent to form the unfinished removing section 222′. The unfinished removing section 222′ is cut in the transverse direction and is bent to form the removing section 222.

After the removing section 222 is formed, the distal end of the removing section 222 and a part of the removing portion 22 that is connected to the groove wall 215 are further polished to be rounded to improve comfort of use and to prevent the user from being injured by accidental touch. Finally, the threaded section 216 is formed on the tubular piece 5, and the manufacturing of the tissue removing member 2 is completed (see FIG. 2). The above description is merely an example of a method for manufacturing the integrally formed tissue removing member 2 of the embodiment. The manufacturing process of the tubular piece 5 may be implemented by a conventional metal processing technology, so that the details thereof are omitted for the sake of brevity, and the person skilled in the art should understand the manufacturing process without further description. In some examples of the embodiment, the rod body 11 may be integrally formed with the tissue removing member 2. In addition, the tissue removing member 2 may be made of polyvinyl chloride (PVC), polyethylene terephthalate (PET), or polymethyl methacrylate (PMMA, also known as acrylic resin) to save on manufacturing costs, thereby making the tissue removing member 2 possible to be disposable after a single use. The tissue removing member 2 may be integrally formed by injection molding or a machine tool.

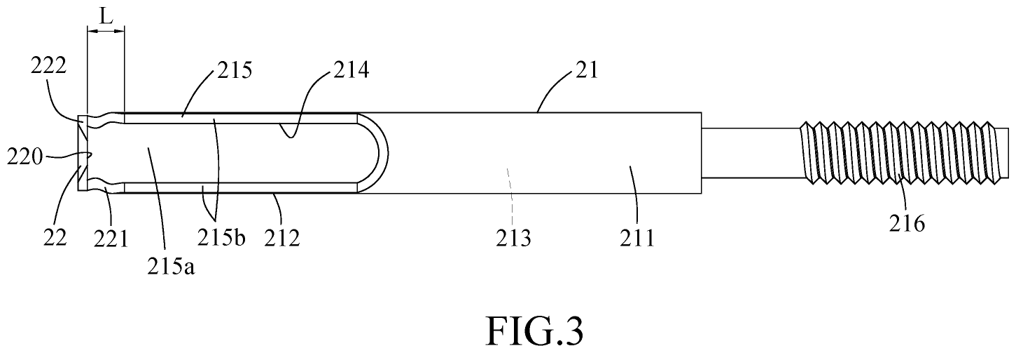

Referring to FIG. 3, it should be noted that, in this embodiment, a length (L) between the removing section 222 and the groove wall 215 in the front-rear direction ranges from 0.1 mm to 1.0 mm, such as from 0.2 mm to 0.4 mm, and thus, when the removing section 222 is used to remove the tissue, resistance from the tissue is smaller and the tissue may be trimmed neatly. Furthermore, an end surface of the tissue removing section 222 may be smooth, serrated, or wavy, but is not limited thereto.

Referring to FIGS. 2 and 4, in this embodiment, the nail tool may further include a cover 4 that is detachably connected to the rod body 11. The cover 4 has a shielding space 41 for accommodating the removing portion 22. Therefore, when not using, the user may connect the cover 4 to the rod body 11 to place the tissue removing member 2 in the shielding space 41, so as to prevent the tissue removing member 2 from being damaged by external forces and from being exposed to moisture or dirt, thereby preventing oxidization or deterioration of the tissue removing member 2.

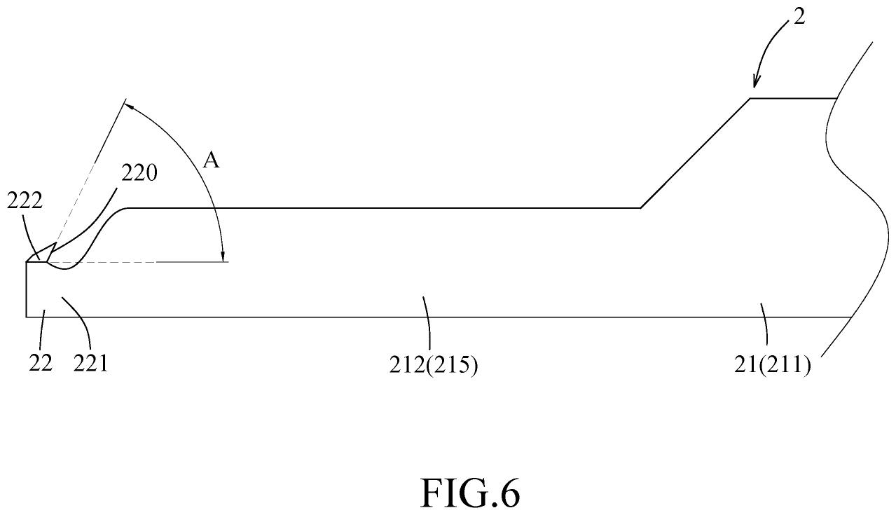

Referring to FIG. 6, in this embodiment, an angle (A) between the rough surface 220 of the removing section 222 and a level line parallel to the front-rear direction ranges from 60 degrees to 90 degrees, such as from 80 degrees to 90 degrees, or from 85 degrees to 90 degrees. FIG. 4 illustrates an example of the embodiment that the angle between the rough surface 220 and the level line is 90 degrees. FIG. 6 illustrates another example of the embodiment that the angle between the rough surface 220 and the level line is smaller than 90 degrees.

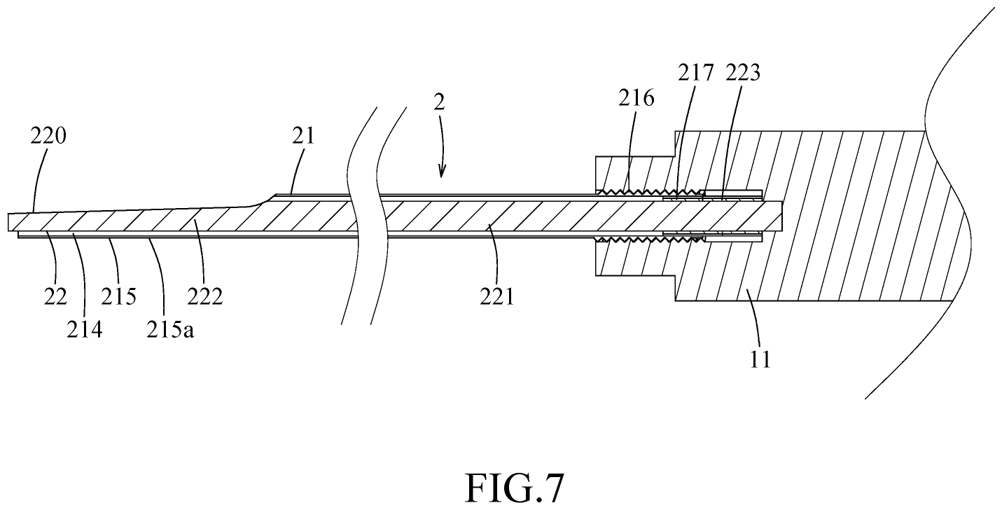

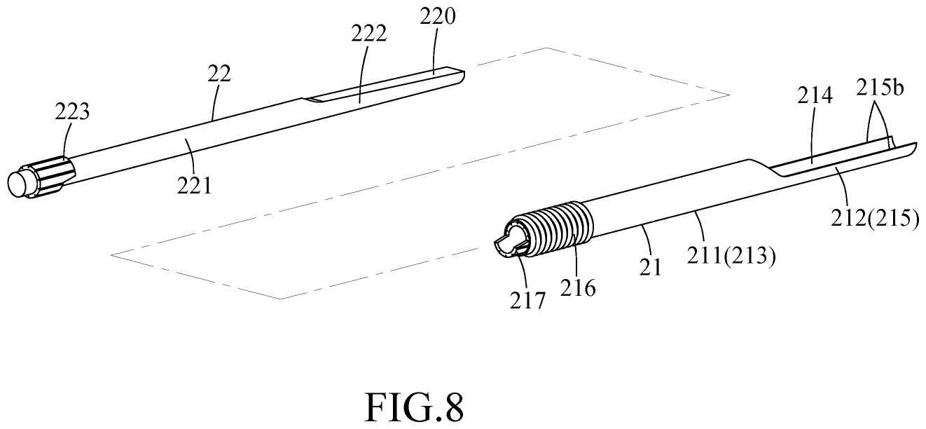

Referring to FIG. 7 and FIG. 8, a second embodiment of the nail tool of the disclosure is illustrated. In the second embodiment, the tissue removing member 2 is two-piece. That is to say, the seat body 21 and the removing portion 22 of the tissue removing member 2 are detachably connected to each other.

In an example of this embodiment, the seat body 21 further has a first assembly portion 217 connected to the connecting section 211. The removing portion 22 includes an extension section 221, a removing section 222 that has the rough surface 220, that is located at the front end of the seat body 21 and that is connected to the extension section 221, and a second assembly portion 223 that surrounds the extension section 221. The removing portion 22 extends into the seat body 21, and the rough surface 220 is exposed from the groove wall 215 of the seat body 21 and faces upward. The removing portion 22 and the seat body 21 are detachably connected to each other through the first assembly portion 217 and the second assembly portion 223. The second assembly portion 223 may correspond to the first assembly portion 217 in shape to allow the removing portion 22 to be positioned with respect to the seat body 21, and to prevent the removing portion 22 from rotating relative to the seat body 32. For example, as shown in FIG. 8, the first assembly portion 217 has a convex portion and a concave portion that are located at a rear end of the first assembly portion 217, and the second assembly portion 223 has a convex portion and a concave portion that are located at a front end of the second assembly portion 223, and that are capable of respectively engaging with the concave portion and the convex portion of the first assembly portion 217, but not limited thereto. That is to say, the first assembly portion 217 and the second assembly portion 223 may have more than one convex portion and more than one concave portion. In an example, the first assembly portion 217 and the second assembly portion 223 have an odd number of the convex portions and an odd number of the concave portions such that shapes of the first assembly portion 217 and the second assembly portion 223 are asymmetric. Therefore, an error during assembly which may cause the rough surface 220 facing downwards may be avoided. It should be noted that the removing portion 22 and the seat body 21 may be detachably connected to each other by, for example, but not limited to, magnetic attraction, screwing, clamping, etc.

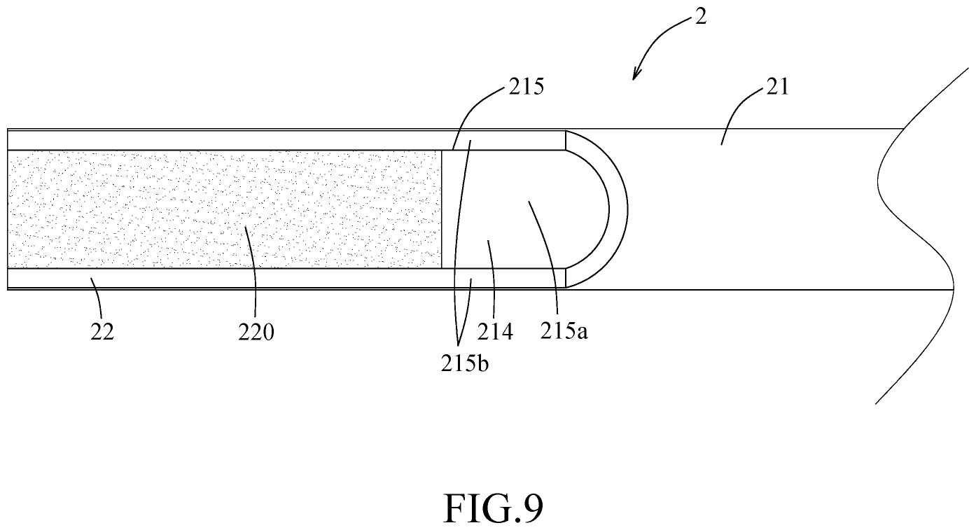

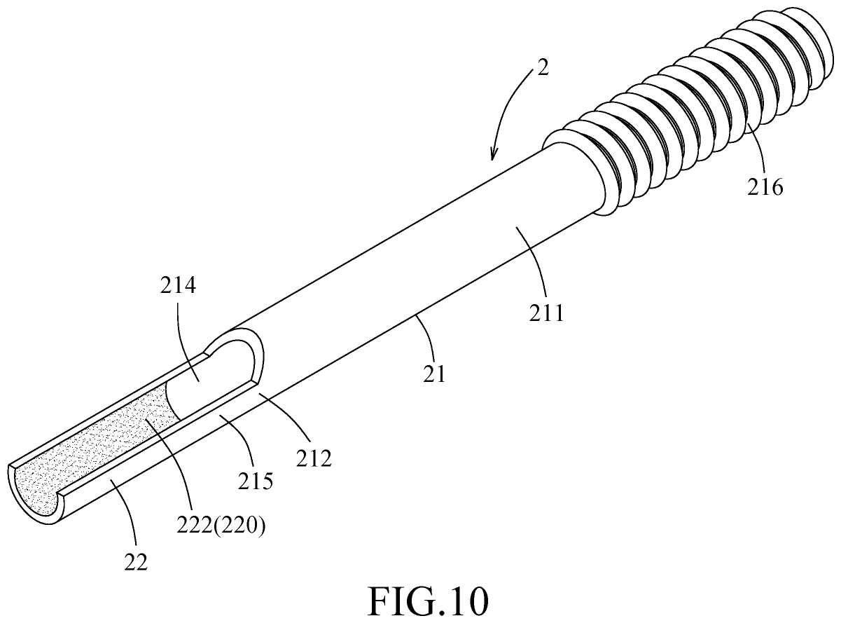

Referring to FIG. 9 and FIG. 10, a third embodiment of the nail tool of the disclosure is illustrated. In the third embodiment, the removing portion 22 includes the removing section 222 that has the rough surface 220. The rough surface 220 is located on the groove wall 215.

The rough surface 220 may be constructed as a series of ridges or abrasives. The rough surface 220 of the removing portion 22 may be formed by directly processing the groove wall 215. Particularly, the rough surface 220 may be formed, but not limited to, by adhering abrasives on the groove wall 215 with glue, or by cutting the groove wall 215 to have a series of ridges. Furthermore, a product that is coated with abrasives (e.g., sandpaper) or that is formed with ridges may be adhered to the groove wall 215 to serve as the rough surface 200.

In summary, according to the disclosure, the groove wall 215 of the nail tool may be used to widen the nail groove to allow the rough surface 200 to extend into the nail groove to remove the tissue, and the tissue thus removed enters the accommodating groove 214. Therefore, the nail tool of the disclosure may remove the tissue in the nail groove easily, so that the tissue may be removed more completely. If a person suffered from the ingrown nail uses the nail tool to treat the ingrown nail on a regular basis, the ingrown nail may be controlled and paronychia may be prevented.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A nail tool for removing tissue in a nail groove adapted for extending into the nail groove to remove the tissue, said nail tool comprising:
a rod body assembly that extends in a front-rear direction and that includes a rod body, said rod body having a front end portion, and a rear end portion that is located opposite to said front end portion; and
a tissue removing member that is connected to said front end portion of said rod body and that includes a seat body and a removing section located at a front end of said seat body, said seat body having a groove wall that defines an accommodating groove, said groove wall having a bottom wall portion, and two side wall portions that extend from said bottom wall portion, said removing section having a rough surface, said side wall portions of said groove wall being adapted to widen the nail groove to allow said rough surface to remove the tissue.

2. The nail tool as claimed in claim 1, further comprising a plug that is disposed in said accommodating groove, said seat body having a connecting section that is connected to said rod body and that defines an accommodating space in spatial communication with said accommodating groove, and a widening section that is located at said front end of said seat body, that is connected to said connecting section, and that has said groove wall, said plug being adapted to prevent the tissue from entering said accommodating space through said accommodating groove.

3. The nail tool as claimed in claim 1, wherein said tissue removing member further includes a removing portion that has said removing section and an extension section connected between a front end of said seat body and said removing section, said removing portion extending forwardly from said groove wall and being bent back to form a hook shape.

4. The nail tool as claimed in claim 1, wherein said rough surface is constructed as a series of ridges or abrasives.

5. The nail tool as claimed in claim 1, wherein said tissue removing member is detachably connected to said rod body.

6. The nail tool as claimed in claim 1, wherein said tissue removing member is integrally formed.

7. The nail tool as claimed in claim 6, wherein said tissue removing member is formed by cutting and bending a tubular piece.

8. The nail tool as claimed in claim 3, wherein said groove wall has an open end, and a closed end that is opposite to said open end in a transverse direction perpendicular to the front-rear direction, a distance between a distal end of said removing section and said closed end of said groove wall in the transverse direction being smaller than a distance between said open end and said closed end of said groove wall in the transverse direction.

9. The nail tool as claimed in claim 8, wherein a distance between the distal end of said removing section and the open end of said groove wall in the transverse direction ranges from 0.1 mm to 1.0 mm.

10. The nail tool as claimed in claim 3, wherein a length between said removing section and said groove wall in the front-rear direction ranges from 0.1 mm to 1.0 mm.

11. The nail tool as claimed in claim 1, wherein said rough surface is located on said groove wall.

12. The nail tool as claimed in claim 1, wherein said rod body is integrally formed with said tissue removing member.

13. The nail tool as claimed in claim 1, wherein said tissue removing member further includes a removing portion that extends into said seat body and that has said removing section, said rough surface of said removing section being exposed from said groove wall.

14. The nail tool as claimed in claim 13, wherein said seat body further has a first assembly portion, said removing portion further having a second assembly portion that corresponds to said first assembly portion in shape to allow said removing portion to be positioned with respect to said seat body.
